(19) United States
(12) Patent Application Publication
(10) Pub. No.: US 2005/0143356 A1
(43) Pub. Date: Jun. 30, 2005

(54) Selective inhibition of cyclooxygenase 1 in the treatment of diabetic nephropathy

(75) Inventor: Matthew Breyer, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/007,060

(22) Filed: Dec. 8, 2004

Related U.S. Application Data
(60) Provisional application No. 60/527,692, filed on Dec. 8, 2003.

(51) Int. Cl. A61K 31/60; A61K 31/401; A61K 31/192; A61K 31/05

(52) U.S. Cl. 514/165; 514/569; 514/570; 514/423; 514/733

(57) ABSTRACT

The present invention relates to the use of COX-1 selective inhibitors to treat diabetic nephropathy. In particular, COX-1 inhibitors may be combined with standard insulin replacment therapy, and/or combined with ACE inhbitor therapy. The therapy is, in a specific embodiment, designed to inhibit systemic COX-1 activity, reflected by inhibition of platelet-stimulated thromboxane production, while not inhibiting macrophage PGE2 production.

This application claims priority to U.S. Provisional Application Ser. No. 60/527,692, filed Dec. 8, 2004, the entire content of which is hereby incorporated by reference.

### BACKGROUND OF THE INVENTION

1. Field of the Invention

The present invention relates generally to the field of human pathology, and more particularly to the area of diabetes. Specifically, it provides for the treatment of diabetic nephropathy by selective inhibition of cyclooxygenase 1. Compositions and methods are disclosed that provide for such therapies, and well as for the screening of potential drugs for this use, are disclosed.

2. Description of Related Art

β-cells of the islets of Langerhans in the pancreas secrete insulin in response to secretagogues such as amino acids, glyceraldehyde, free fatty acids, and, most prominently, glucose. Increased insulin secretion in response to a glucose load prevents hyperglycemia in normal individuals by stimulating glucose uptake into peripheral tissues, particularly muscle and adipose tissue. Individuals who lack proper insulin/glucose regulation suffer from diabetes.

Insulin-dependent diabetes mellitus, or IDDM (also known as Juvenile-onset or Type I diabetes), represents approximately 10% of all human diabetes. IDDM is distinct from non-insulin dependent diabetes (NIDDM) in that only IDDM involves primary or initial destruction of the insulin producing β-cells of the islets of Langerhans. The destruction of β-cells in IDDM appears to be a result of specific autoimmune attack, in which the patient's own immune system recognizes and destroys the β-cells, but not the surrounding α-cells (glucagon producing) or δ-cells (somatostatin producing) that comprise the islet.

Type II diabetes, in contrast to type I, appears to arise at least in part from the inability of cells to respond to insulin. Insulin is responsible for stimulating glucose uptake into its target cells by a process which involves the translocation of the GLUT4 isoform of glucose transporter from an intracellular vesicular compartment(s) to the plasma membrane. Thus, despite an ability to sense glucose and send proper signals (insulin) for glucose uptake, the afflicted individuals nonetheless suffer from poor glucose clearance and storage. The pathways that are responsible for this faulty insulin response unfortunately remain obscure.

One of the most serious complications of diabetes is diabetic nephropathy (DN), a microvascular abnormality characterized by albuminuria and deterioration of normal renal function to end stage renal disease (ESRD). Though drug and dietary treatments can help control DN, there remains a need for new and improved therapies for this syndrome.

### SUMMARY OF THE INVENTION

Thus, in accordance with the present invention, there is provided a method for inhibiting or treating diabetic nephropathy comprising administering to a subject having diabetes in need thereof a cyclooxygenase 1 (COX-1) selective inhibitor. The administration may be chronic or long-term administration. The COX-1 selective inhibitor may be provided at a dosage sufficient to inhibit platelet stimulated thromboxane production, but not sufficient to inhibit activated macrophage PGE2 production. COX-1 selective inhibitors include SC 560 (SC58560), ketorolac, aspirin, valeryl salicylate, resveratrol, flurbiprofen, fenoprofen, sulindac, piroxicam, ibuprofen, indomethacin, naproxen, oxaprosin, tenoxicam, and tolmetin. Inhibiting or treating may comprise blocking or reducing a diabetes-related increase in proteinuria and/or blocking or reducing a diabetes-related drop in glomerular filtration rate. The administration may be oral. The method may further comprise co-administering an angiotensin converting enzyme inhibitor. The subject may suffer from type I diabetes or type II diabetes.

In another embodiment, there is provided an improved method of type I diabetes therapy comprising chronic administration to a diabetic subject of a composition comprising insulin and a cyclooxygenase (COX-1) selective inhibitor. The administration may be intramuscular, intravenous, subcutaneous, intranasal, inhalational or transdermal. The said COX-1 selective inhibitor may be provided at a dose providing at least 2-fold, including 10-fold, greater in vivo activity against COX-1 and compared to COX-2. The COX-1 selective inhibitor may be SC 560, ketorolac, aspirin, valeryl salicylate, resveratrol, flurbiprofen, fenoprofen, sulindac, piroxicam, ibuprofen, indomethacin, naprosen, oxaprosin, tenoxicam, and tolmetin.

Also encompassed by the present invention are pharmaceutical formulations comprising (a) insulin and a COX-1 selective inhibitor, and (b) an ACE inhibitor and a COX-1 selective inhibitor.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—Histological Alterations in Kidney of Diabetic C57BLKS db/db Mice Treating with COX-1 Inhibitor. Periodic acid-Schiff stain (original magnification, ×200) shows normal appearance in outer cortex of 24-week-old control db/+ mouse (FIG. 1A) and increase in mesangial matrix and glomerular hypertrophy in diabetic db/db mouse (FIG. 1B). In contrast (FIG. 1C), the mesangial expansion is less pronounced in COX-1 inhibitor treated mouse.

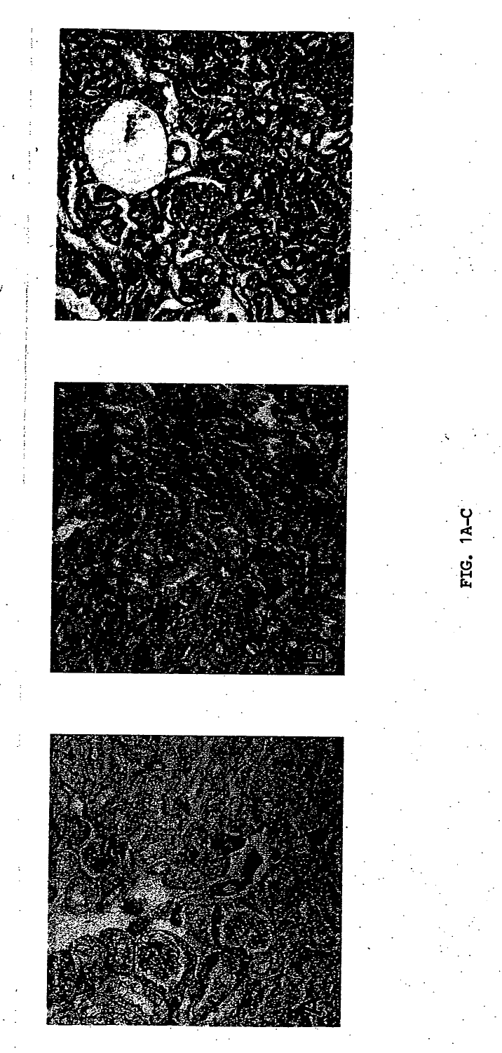

### DESCRIPTION OF ILLUSTRATIVE EMBODIMENTS

In light of the need for alternative therapeutic strategies for treating diabetic nephropathy (DN), the present inventor has determined that chronic treatment with a selective inhibitor of the cyclooxygenase 1 enzyme provides long-term benefits to kidney function. Using a murine model for type II diabetes (db/db), after 6 months of treatment, albuminuria was significantly reduced in treated versus untreated animals. This occurred despite the fact that the COX-1 inhibitor did not affect body weight gain, blood sugar or Hb1C in diabetic mice versus vehicle treated controls. COX-1 treated mice exhibited a significantly greater glomerular filtration rate (GFR) than untreated mice. Previous studies demonstrated that COX-2 inhibition may reduce GFR and renal blood flow, whereas the present study suggests that COX-1 inhibition is relatively renal sparing—actually improving glomerular filtration rate. Moreover, in untreated mice, histopathology revealed severe mesangial expansion and, thickening of Bowman's capsule. In contrast, the mesangial expansion was less pronounced in COX-1 inhibitor-treated mice. Together, these data indicate that chronic COX-1 inhibition provides an alternative therapeutic approach to preserve renal function, reduce proteinuria and mesangial expansion in diabetic nephropathy.

### I. CYCLOOXYGENASES AND INHIBITORS THEREOF

In 1971, Vane published his seminal observations proposing that the ability of NSAIDs to suppress inflammation rests primarily on their ability to inhibit the cyclooxygenase (COX) enzyme. Given this connection, NSAIDs have been used for the past 25 years to inhibit the COX enzyme and the eicosanoids derived from this pathway in normal and abnormal physiologic states.

Prostaglandins are formed by the oxidative cyclization of the central 5 carbons within 20 carbon polyunsaturated fatty acids. The key regulatory enzyme of this pathway is COX (COX) (PGH synthase), which catalyzes the conversion of arachidonic acid (or other 20 carbon fatty acids) to prostaglandin (PG) G₂ and PGH₂. PGH₂ is subsequently converted to a variety of eicosanoids that include PGE₂, PGD₂, PGF₂, PGI₂, and thromboxane (TX) A₂. The array of PGs produced varies depending on the downstream enzymatic machinery present in a particular cell type. All NSAIDs in clinical use have been shown to inhibit COX, leading to a marked decrease in PG synthesis.

PGs play a central role in inflammation, and also regulate other critical physiological responses such as blood clotting, ovulation, initiation of labor, bone metabolism, nerve growth and development, wound healing, kidney function, blood vessel tone, and immune responses. PGs are synthesized in a broad range of tissue types and serve as autocrine or paracrine mediators to signal changes within the immediate environment. Unfortunately, in light of the broad role PGs play in normal human physiology, systemic suppression of PG synthesis through inhibition of COX can lead to unwanted side effects, including gastro-intestinal ulceration, high blood pressure, edema and kidney failure.

Using an anti-COX antibody, two different COX isoforms were identified. These are now designated as COX-1 and COX-2, which are derived from distinct genes that diverged well before birds and mammals. Studies revealed that while both enzymes carry out essentially the same catalytic reaction and have similar primary protein structures, many of the inflammatory, inducible effects of COX appeared to be mediated by COX-2, while many of the housekeeping effects appear to be mediated by COX-1. The functional role for each isoform is consistent with tissue expression patterns.

A. COX-1

COX-1 has been localized in nearly all tissues under basal conditions, and thus one would expect that this enzyme's primary function is to provide PG precursors for homeostatic regulation. One important site of COX-1 function is the blood platelet, where the enzyme is responsible for providing precursors for thromboxane synthesis. Since platelets cannot produce an inducible enzyme in response to activating conditions, they instead carry a supply of COX-1. In the presence of an NSAID like aspirin, platelets are prevented from generating thromboxane during activation and fail to complete successful aggregation, inhibiting their thrombogenic potential. In the adjacent vascular endothelium, PGs play a different role. The release of eicosanoids by activated platelets is thought to provide both a substrate and stimulus for the generation of anti-thrombogenic prostacyclin (PGI₂) by the endothelium. This compound stimulates vasodilatation, counteracting the vasoconstrictor, thromboxane.

COX-1-derived prostanoids appear to function in other physiological systems leading to contractile conditions: in both the kidney and the stomach. During times of lowered blood volume, the kidney releases angiotensin and other factors to maintain blood pressure by systemic vasoconstriction. At the same time, angiotensin invokes PG synthesis in the kidney. COX-1 is expressed in the vasculature, glomeruli, and collecting ducts of the kidney, and it appears to be important in producing PGs, which maintain renal plasma flow and glomerular filtration rate during conditions of systemic vasoconstriction. In the presence of NSAIDs, this protective response fails, leading to renal ischemia and damage in susceptible individuals. In the gastric antrum, NSAID use leads to ischemia which contributes to mucosal damage and ulceration. The enzyme blocked by NSAIDs is thought to be COX-1 that produces PGs, which alter blood flow in the microcirculation of the gastric mucosa.

B. COX-2

After the discovery of the second isoform of COX, a screen of existing NSAIDs was conducted to determine differential effects on inhibition of COX-1 versus COX-2. Interestingly, some were found to have a 20- to 70-fold selective preference. This permitted one to use differential inhibition of COX-1 or COX-2 activities to sort out the relative contributions of these isoforms. While initial studies upheld the concept that COX-2 is mainly an inflammatory, inducible enzyme, more recent studies are beginning to reveal additional functions.

For example, prostaglandins are known to serve as important physiologic modulators of vascular tone and sodium and water homeostasis in the mammalian kidney, including modulation of glomerular hemodynamics, tubular reabsorption of sodium and water, and regulation of renin secretion. While COX-1 has long been recognized to be involved in normal kidney function, COX-2 is now seen to have a distinct role providing vasodilator prostanoids. COX-2 also seems to have some role in maintaining gastrointestinal integrity, ovarian and uterine function, bone metabolism, inflammation and arthritis, pain, cancer, neuronal function and possibly even the development of Alzheimer's Disease.

C. COX-1 Selective Inhibitors

A variety of COX-1 inhibitors have been identified. Some of these, like SC 560 are selective inhibitors, while others may exhibit dose-dependent selectivity, such as ketorolac, aspirin, valeryl salicylate, resveratrol, flurbiprofen, fenoprofen, sulindac, piroxicam, ibuprofen, indomethacin, naproxen, oxaprosin, tenoxicam, and tolmetin.

### II. DIABETIC NEPHROPATHY

Diabetic nephropathy (DN) is a microvascular complication of diabetes. If is characterized by albuminuria (excessive urine albumin excretion), with ultimate progression to end stage renal disease (ESRD). There are over 7 million diabetes patients in the U.S.—about 30-40% of those with type I diabetes, and 5-60% of those with type II diabetes (depending on ethnicity), will develop DN. The estimated cost of treating DN is about $20,000,000,000 per year. Risk factors include hypertension, hyperglycemia, microalbuminuria, duration of diabetes, ethnicity, male gender, smoking and possibly hyperlipidemia.

The natural history of diabetic nephropathy is well established. Stage 1 is characterized by hyperfiltration (glomerular filtration rate (GFR) increased in both type I and II). Stage 2, sometimes called the “silent stage,” shows a decline in GFR back to normal, although some type II patients may begin to exhibit increased albumin excretion and hypertension. Stage 3, referred to as “incipient diabetes,” is characterized by microalbuminuria (30-300 mg/day), falling GFR (below normal), and elevated blood pressure in type I patients. Stage 4, or “overt diabetic nephropathy,” is characterized by over albuminuria, reduced GFR and hypertension. Finally, in Stage 5, patients suffer from extremely low GFR, hypertension and ultimately ESRD.

A number of approaches to controlling DN have been proposed. First and foremost, as with the treatment of diabetes generally, proper glycemic control helps limit DN. Second, ACE inhibitors (and some calcium channel blockers) have been utilized to decrease albumin excretion. Other approaches are to reduce protein intake and to control blood pressure by various means. A final option is pancreatic transplant.

### III. SCREENING METHODS

The present invention also contemplates the screening of COX-1 inhibitors for their effects against diabetic nephropathy. In particular aspects, the screen may be designed to test for the ability to inhibit platelet stimulated thromboxane production, but not inhibit LPS-activted macrophage PGE2 production. The methods may further identify compounds on the basis of their ability to block or reduce a diabetes-related increase in proteinuria and/or block or prevent the diabetes-related drop in glomerular filtration rate.

A. In Cyto Assays

In accordance with the present invention, screening of COX-1 inhibitors may be undertaken using cell lines, particularly those that permit measurement of COX-1-stimulated thromboxane production and COX-2-derived macrophage PGE2 production. HEK293 cells transfected with rabbit or human cyclooxygenase may also be tested.

Assays for platelet-stimulated thromboxane production can be performed using commercially available kits, such as the Thromboxane B2 EIA Kit from Cayman Chemical. The limit of detection is 13 pg/ml. Similar kits—TxB₂ Correlate-EIA Kit, TxB₂ Correlate-CLIA Kit and Immunoassay Kit—are available from Assay Designs, Inc. PGE₂ and TxB2 may also be assayed using gas chromatograph-mass spectroscopy.

B. In Vivo Assays

The present invention particularly contemplates the use of various animal models for the testing of COX-1 selective inhibitors. In particular, murine models for diabetic nephropathy, including the C57BLKSdb/db and KK mouse models, are used. Models of Type I diabetes include streptozotocin-treated animals and mice with a mutation in the Ins2 gene (Akita mice) which are commercially available (Jackson Labs). Phenotypes of interest are the blocking or reduction of a diabetes-related increase in proteinuria, and/or blocking or reduction of a diabetes-related drop in glomerular filtration rate. Such assays are standard in the field and well known to those of skill in the art.

Other models of diabetes may also prove useful. B6.HRS(BKS)-Cpe^{fat}/+ (Jackson Labs) is a C57BL/6J congenic strain carrying the fat spontaneous mutation. B6.HRS(BKS)-Cpe^{fat}/+ mice have been backcrossed to C57BL/6J for 10 generations (N10). Homozygous Cpe^{fat} mice develop a diabetic phenotype characterized by hyperglycemia and insulin resistance. C57BL/6J mutant mice also develop obesity at an earlier age than BKS.HRS-Cpe^{fat}/J mice (Jackson Labs), with the females becoming heavier than males. Obesity develops later than in obese (B6.V-Lep^{ob}; Jackson Labs) and diabetes (BKS.Cg-m +/+ Lepr^{db}; Jackson Labs) mutant mice. Cpe^{fat} mice actually weigh less than wildtype controls prior to weaning age. Weide & Lacy (1991); Naggert et al., (1995). C57BL/6-Ins2^{Akita} (Jackson Labs) is another diabetes model associated with proinsulin processing defects.

### IV. THERAPIES

A. Methods of Treatment

In accordance with the present invention, methods of treatment of diabetic nephropathies are provided. The methods rely on the long-term provision of a COX-1 selective inhibitor. The amounts and frequency of administration currently recommended for COX-1 inhibitors such as ketorolac are believed to be appropriate treatment regimens. Generally, the desired dose/frequency is sufficient to inhibit 80% of tissue COX-1 activity, but less than 10% of tissue COX-2 activity over a period of 24 hours.

It may prove useful to rotate different COX-1 inhibitors as part of the therapy. Thus, for example, one may select two, three or more inhibitors, with the inhibitor in use being switched every 2-8 weeks. It may also be desirable to combine two or more COX-1 inhibitors, possibly in reduced dosages, to gain a more effective therapeutic effect.

B. Pharmaceutically Acceptable Formulations

Where clinical applications are contemplated, it will be necessary to prepare pharmaceutical compositions of the present invention in a form appropriate for administration to a subject. The compositions will generally be prepared as essentially free of pyrogens, as well as other impurities that could be harmful to humans or animals.

One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Under ordinary conditions of storage and use, pharmaceutical preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of a therapeutic composition is determined based on the intended goal. The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

C. Route of Administration

Virutally any route of administration may be used, although it is envisioned that COX-1 inhibitor oral administration is by far the most straightforward route. Alternatively, the COX-1 inhibitors of the present invention can be administered intravenously, transdermally, intradermally, intraarterially, intraperitoneally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

D. Adjunct Therapies and Related Procedures

In accordance with the present invention, it may prove advantageous to combine the methods disclosed herein with adjunct therapies or procedures to enhance the overall therapeutic effect. Such therapies and procedures are set forth in general, below. A skilled physician will be apprised of the most appropriate fashion in which these therapies and procedures may be employed.

i. Supplemental Insulin Therapy

The present invention, though designed to eliminate the need for other therapies, should work well in combination with traditional insulin supplementation. Such therapies should be tailored specifically for the individual patient given their current clinical situation, and particularly in light of the extent to which transplanted cells can provide insulin. The following are general guidelines for typical a “monotherapy” using insulin supplementation by injection.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually prebreakfast when the child may be in a hurry to go to school) and the thigh when the patient can wait 30 minutes after injection for his meal (usually predinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2,3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hyopglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken—one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus manner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

Also contemplated are the use of implantable insulin pumps, intranasal and transdermal insulin administration, small molecule and insulin mimetics.

ii. ACE Inhibitors

ACE inhibitors, or angiotensin converting enzyme inhibitors reduce peripheral vascular resistance via blockage of the angiotensin converting enzyme. This action reduces the myocardial oxygen consumption, thereby improving cardiac output and moderating left ventricular and vascular hypertrophy. ACE inhibitors are essential for treatment of CHF due to systolic dysfunction.

In one aspect of the invention, COX-1 inhibitors may be administered with an ACE inhibitor, such as Accupril (quinapril), Aceon (perindopril), Altace (ramipril), Capoten (captopril), Lotensin (benazepril), Mavik (trandolapril), Monopril (fosinopril), Prinivil (lisinopril), Univasc (moexipril), Vasotec (enalaprilat, enalapril), Zestril (lisinopril).

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The C57BLKS db/db mice is a rodent model of type II diabetes mellitus. A LepR^{db} (db) mutation on chromosome 4 (G→T point mutation) has been identified in the leptin receptor gene. This mutation generates a new donor splice site, leading to prematurely termination of the the intracellular domain of leptin receptor and lack of leptin signal transduction. C57BLKS db/db strain is more susceptible to the development of diabetic nephropathy than the C57BL/6J db/db strain. Animals are characterized by obesity, insulin resistance, diabetes and diabetic nephropathy (DN). In the setting of hyperglycemia, reduced glomerular filtration rate (˜50% of that in age, gender, strain-matched controls) is observed, along with albuminuria (approximately 10-100 fold greater that present in normal mice of same age, strain, and gender). Pathologic changes include mesangial matrix expansion (>50% increase in the majority of glomeruli) and GBM thickening >25%, absence of GBM electron dense material by EM, arteriolar hyalinosis (any degree) and tubulo-interstitial fibrosis.

C57BLKS db/db mice were administered COX-1 selective inhibitor SC58560 (15 mg/ml in 95% polyethylene glycol 200 and 5% Tween-20) diluted 1:500 in tap water and given in the drinking water. Inhibitor was given for 6 months, while the control diabetic mice were given vehicle alone (n=8 per group). Blood samples were drawn for analysis of glucose and glycosylated hemoglobin (HbA1c) monthly. The urine samples were collected monthly and albumin/creatinine ratio was determined by enzyme linked immunosorbent assays (Exocell Inc, PA, USA). Glomerular filtration rate (GFR) was determined using FITC-inulin clearance based on plasma elimination rate following single bolus intravenous injection.

### Example 2

### Results

The relative abundance of cyclooxygenase-2 (COX-2) in the kidney and clinical availability of selective COX-2 inhibitors has motivated numerous recent studies addressing the role of COX-2 in renal diseases including diabetic nephropathy. Cyclooxygenase-1 (COX-1) is also abundantly expressed in the kidney, however its role in health and disease remains poorly defined. To evaluate the functional importance of the altered COX-1 production in the onset of diabetic nephropathy, the inventors studied the effect of chronic administration of the selective COX-1 inhibitor (SC8560) in the C57BLKS db/db mice(db/db), a rodent model of type II diabetes mellitus. Diabetic mice were provide the COX-1 inhibitor for 6 months, while control diabetic mice given vehicle alone. After 6 months of treatment, albuminuria as assessed by albumin/creatinine ratio was significantly reduced in treated vs. untreated mice (77±49 vs. 342±38 μg/mg, p<0.005). This occurred despite the fact that COX-1 inhibitor did not affect the body weight gain, blood sugar and Hb1C in diabetic mice vs. vehicle treated controls. Glomerular filtration rate was also determined using FITC-inulin clearance. COX-1 inhibitor treated mice exhibited a significantly greater GFR than untreated db/db mice (462±46 vs. 298±35 ul/min/mouse, p<0.01, n=8 in each group). Since previous studies demonstrated COX-2 inhibitor may reduce GFR and renal blood flow, the present studies suggest COX-1 inhibition is relatively renal sparing—actually improving glomerular filtration rate. In untreated mice histopathology revealed severe mesangial expansion and, thickening of Bowman's n capsule. In contrast, the mesangial expansion was less pronounced in COX-1 inhibitor treated mice. These data suggest that chronic COX-1 inhibition might provide an approach to preserve renal function, reduce proteinuria and mesangial expansion in diabetic nephropathy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
U.S. Pat. No. 4,415,732
U.S. Pat. No. 4,458,066
U.S. Pat. No. 4,554,101
U.S. Pat. No. 4,683,195
U.S. Pat. No. 4,683,202
U.S. Pat. No. 4,800,159
U.S. Pat. No. 4,873,191
U.S. Pat. No. 4,883,750
U.S. Pat. No. 5,279,721
U.S. Pat. No. 5,354,855
U.S. Pat. No. 5,792,453
U.S. Pat. No. 5,795,715
U.S. Pat. No. 5,795,715
U.S. Pat. No. 5,889,136
U.S. Pat. No. 6,100,242
Weide & Lacy, Hereditary hydronephrosis in C57BL/KsJ mice. Lab Anim Sci 1991; 41:415-418.
Naggert et al., Hyperproinsulinaemia in obese fat/fat mice associated with a carboxypeptidase E mutation which reduces enzyme activity. Nat Genet 1995; 10:135-142.
Altschul et al., J. Mol. Biol., 215(3):403-410, 1990.
Baichwal et al., In: Gene transfer, Kucherlapati (Ed), Nyork, Plenum Press, 117-148, 1986.
Barany and Merrifield, In: The Peptides, Gross and Meienhofer (Eds.), Academic Press, New York, 1-284, 1979.
Benvenisty and Neshif, Proc. Natl. Acad. Sci. USA, 83(24):9551-9555, 1986.
Bonangelino et al., Mol. Cell Biol., 17(12):6847-6858, 1997.
Bosher and Labouesse, Nat. Cell Biol., 2:E31-E36, 2000.
Brinster et al., Proc. Natl. Acad. Sci. USA, 82(13):4438-4442, 1985.
Bryant et al., J. Cell Biol., 142(3):651-653, 1998.
Cantley and Neel, Proc. Natl. Acad. Sci. USA, 96(8):4240-4245, 1999.
Capaldi et al., Biochem. Biophys. Res. Comm., 74(2):425-433, 1977.
Caplen et al., Gene, 252(1-2):95-105, 2000.
Chang et al., Hepatology, 14:134A, 1991.
Chen and Okayama, Mol. Cell Biol., 7:2745-2752, 1987.
Chen et al., J. Biol. Chem., 272(43):27401-27410, 1997.
Coffin, In: Virology, Fields et al. (Eds.), NY, Raven Press, 1437-1500, 1990.
Conradt et al., J. Cell Biol., 119(6):1469-1479, 1992.
Cooke et al., Curr. Biol., 8(22):1219-1222, 1998.
Couch et al., Am. Rev. Resp. Dis., 88:394-403, 1963.
Coupar et al., Gene, 68:1-10, 1988.
Cowles et al., Cell, 91(1):109-118, 1997.
Cremona and De Camilli, J. Cell Sci., 114(Pt6):1041-1052, 2001.
Cullen et al., Curr. Biol., 11(21):R882-893, 2001.
Culliton, Science 246:746-751, 1989.
Davis et al., Meth. Enzymol., 65:404-411, 1980.
Dove et al., Curr. Biol., 12(11):885-893, 2002.
Dove et al., Nature, 390(6656):187-192, 1997.
Elbashir et al., Nature, 411(6836):494-498, 2001.
EPO 0 273 085
Fechheimer et al., Proc. Natl. Acad. Sci. USA, 84:8463-8467, 1987.
Ferkol et al., FASEB J., 7:1081-1091, 1993.
Fire et al., Nature, 391:806-811, 1998.
Forster and Symons, Cell, 49(2):211-220, 1987.
Foti et al., Mol. Biol. Cell, 12(8):2396-411, 2001.
Fraley et al., Proc. Natl. Acad. Sci. USA, 76:3348-3352, 1979.
Freifelder, In: Physical Biochemistry Applications to Biochemistry and Molecular Biology, 2Ed. Wm. Freeman and Co., NY, 1982.
Friedmann, Science, 244:1275-1281, 1989.
Gary et al., J. Cell Biol, 143(1):65-79, 1998.
Gerlach et al., Nature, 328:802-805, 1987.
Ghosh and Bachhawat, In: Liver diseases, targeted diagnosis and therapy using specific receptors and ligands, Wu and Wu (Eds.), NY, Marcel Dekker, 87-104, 1991.
Ghosh-Choudhury et al., EMBO J., 6:1733-1739, 1987.
Gomez-Foix et al., J. Biol. Chem., 267:25129-25134, 1992.
Gopal, Mol. Cell Biol., 5:1188-1190, 1985.
Graham and Prevec, Biotechnology, 20:363-390, 1992.
Graham and Prevec, In: Methods in Molecular Biology: Gene Transfer and Expression Protocol, Murray (Ed.), Clifton, N.J., Humana Press, 7:109-128, 1991.
Graham and van der Eb, Virology, 52:456-467, 1973.
Graham et al., J. Gen. Virol., 36:59-72, 1977.
Grishok et al., Science, 287:2494-2497, 2000.
Grunhaus and Horwitz, Seminar in Virology, 3:237-252, 1992.
Guo et al., Bioorg. Med. Chem. Lett., 9:419, 1999.
Guo et al., J. Biol. Chem., 274:12990-995, 1999.
Hardie et al., Neuron, 30(1):149-159, 2001.
Harland and Weintraub, J. Cell Biol., 101:1094-1099, 1985.
Hawkins et al., Biochem J., 238(2):507-516, 1986.
Hermonat and Muzyczka, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hogan et al., In: Manipulating the Mouse Embryo: A Laboratory Manual, 2nd ed.,Cold Spring Harbor Laboratory Press, 1994.
Horwich et al. J. Virol., 64:642-650, 1990.
Huang et al., Nature,391(6669):803-806, 1998.
Hughes et al., J. Biol. Chem., 275(2):801-8, 2000.
Huijbregts et al., Traffic, 1(3):195-202, 2000.
Ikonomov et al., J. Biol. Chem., 2:2, 2001.
Innis et al., PCR Protocols, Academic Press, Inc., San Diego Calif., 1990.
Itoh et al., J. Biol. Chem., 275(25):19389-19394, 2000.
Janez et al., Endocrinology, 141(12):4657-4663, 2000a.
Janez et al., J. Biol. Chem., 275(35):26870-26876, 2000b.
Johnson et al., In: Biotechnology and Pharmacy, Pezzuto et al., Eds., Chapman and Hall, New York, 1993.
Joly et al., J. Biol. Chem., 270(22):13225-13230, 1995.
Jones and Shenk, Cell, 13:181-188, 1978.
Jones et al., J. Biol., 274(26):18407-18413, 1999.
Joyce, Nature, 338:217-244, 1989.
Kaneda et al., Science, 243:375-378, 1989.
Karlsson et al., EMBO J., 5:2377-2385, 1986.
Kato et al., J. Biol. Chem., 266:3361-3364, 1991.
Ketting et al., Cell, 99:133-141, 1999.
Kim and Cook, Proc. Natl. Acad. Sci. USA, 84(24):8788-8792, 1987.
Klein et al., Nature, 327:70-73, 1987.
Kyte and Doolittle, J Mol Biol, 157(1):105-32, 1982.
Lanier and Gertler, Curr. Biol., 10(18):R655-657, 2000.
Le Gal La Salle et al., Science, 259:988-990, 1993.
Levrero et al., Gene, 101: 195-202, 1991.
Li et al., J. Biol. Chem., 276(25):22883-22891, 2001.
Lin et al., Nature, 402:128-129, 1999.
Macejak and Sarnow, Nature, 353:90-94, 1991.
Maehama and Dixon, J. Biol. Chem., 273(22):13375-13378, 1998.
Mann et al., Cell, 33:153-159, 1983.
Markowitz et al., J. Virol., 62:1120-1124, 1988.
Marshall et al., J. Biol. Chem., 271:183, 1996.
McEwen et al., J. Biol. Chem., 274(48):33905-33912, 1999.
Merrifield, Science, 232: 341-347, 1986.
Michel and Westhof, J. Mol. Biol., 216:585-610, 1990.
Montgomery et al., Proc. Natl. Acad. Sci. USA, 95:155-2-15507, 1998.
Nicolas and Rubinstein, In: Vectors: A survey of molecular cloning vectors and their uses, Rodriguez and Denhardt (Eds.), Stoneham, Butterworth, 494-513, 1988.
Nicolau and Sene, Biochim. Biophys. Acta, 721:185-190, 1982.
Nicolau et al., Methods Enzymol., 149:157-176, 1987.
Palmiter et al., Nature, 300:611, 1982.
Paskind et al., Virology, 67:242-248, 1975.
Pattni et al., Curr. Biol., 11(20):1636-1642, 2001.
PCT App. WO 00/44914
PCT App. WO 01/36646
PCT App. WO 01/68836
PCT App. WO 90/07641
PCT App. WO 99/32619
Pelletier and Sonenberg, Nature, 334:320-325, 1988.
Perales et al., Proc. Natl. Acad. Sci. USA, 91:4086-4090, 1994.
Pignon et al., Hum. Mutat., 3:126-132, 1994.
Potter et al., Proc. Natl. Acad. Sci. USA, 81:7161-7165, 1984.
Racher et al., Biotech. Tech., 9:169-174, 1995.
Ragot et al., Nature, 361:647-650, 1993.
Reggiori and Pelham, Nat. Cell Biol., 4:117-23, 2002.
Reinhold-Hurek and Shub, Nature, 357:173-176, 1992.
Renan, Radiother. Oncol., 19:197-218, 1990.
Rich et al., Hum. Gene Ther., 4:461-476, 1993.
Ridgeway, In: A survey of molecular cloning vectors and their uses, Rodriguez et al. (Eds.), Stoneham, Butterworth, 467-492, 1988.
Rippe et al., Mol. Cell Biol., 10:689-695, 1990.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sambrook et al., “Molecular Cloning,” A Laboratory Manual, 2d Ed., Cold Spring Harbor Laboratory Press, New York, 13.7-13.9:1989.
Sarver et al., Science, 247:1222-1225, 1990.
Sbrissa et al., Biochemistry, 39(51):15980-15989, 2000.
Sbrissa et al., J. Biol. Chem., 274(31):21589-21597, 1999.
Scanlon et al., Proc. Natl. Acad. Sci. USA, 88:10591-10595, 1991.
Schafer, Curr. Opin. Cell Biol., 14(1):76-81, 2002.
Schu et al., Science, 260(5104):88-91, 1993.
Sharp et al., Science, 287:2431-2433, 2000.
Sharp Genes Dev., 13:139-141, 1999.
Sherman, Methods Enzymol., 194:3-21, 1991.
Shisheva et al., J. Biol. Chem., 276(15):11859-11869, 2001.
Shisheva et al., Mol. Cell Biol., 19(1):623-634, 1999.
Sikorski and Hieter, Genetics, 122(1):19-27, 1989.
Simonsen et al., Curr. Opin. Cell Biol., 13(4):485-492, 2001.
Srinivasan et al., Eur. J. Cell Biol., 74(4):350-60, 1997.
Stack et al., Annu. Rev. Cell Dev. Biol., 11:1-33, 1995.
Stewart and Young, In: Solid Phase Peptide Synthesis, 2Ed., Pierce Chemical Co., 1984.
Stolz et al., J. Biol. Chem., 273(19):11852-61, 1998a.
Stolz et al., Genetics, 148(4):1715-29, 1998b.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene Ther., 1:241-256, 1990.
Sui et al., Proc. Natl. Acad. Sci. USA, 95(3):1307-1312, 1998.
Tabara et al, Cell, 99:123-132, 1999.
Tam et al., J. Am. Chem. Soc., 105:6442, 1983.
Taylor et al., Proc. Natl. Acad. Sci. USA, 97(16):8910-8915, 2000.
Temin, In: Gene Transfer, Kucherlapati (Ed.), NY, Plenum Press, 149-188, 1986.
Toker and Cantley, Nature, 387(6634):673-676, 1997.
Top et al., J. Infect. Dis., 124:155-160, 1971.
Tur-Kaspa et al., Mol. Cell Biol., 6:716-718, 1986.
Urbanowski et al., Traffic, 2:622-39, 2001.
Vancurova et al. J. Biol. Chem., 274(2):1147-1155, 1999.
Varmus et al., Cell, 25:23-36, 1981.
Vida and Emr, J. Cell Biol., 128(5):779-792, 1995.
Vieira et al., J. Cell Biol., 155(1):19-25, 2001.
Wagner et al., Science, 260:1510-1513, 1990.
Wang et al., J. Cell Biol., 140(5):1063-1074, 1998.
Wang et al., Science, 274, 784-787, 1996.
Weisman et al., J. Cell Biol., 105:1539-1547, 1987
Whiteford et al., Biochem J., 319(Pt3):851-860, 1996.
Whiteford et al., Biochem. J., 323(Pt3):597-601, 1997.
Wincott et al., Nucleic Acids Res., 23(14):2677-2684, 1995.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., J. Virol., 68:4847-4856, 1994a.
Yang et al., Proc. Natl. Acad. Sci. USA, 87:9568-9572, 1990.
Zelenin et al., FEBS Lett., 280:94-96, 1991.

1. A method for inhibiting or treating diabetic nephropathy comprising administering to a subject having diabetes in need thereof a cyclooxygenase 1 (COX-1) selective inhibitor.

2. The method of claim 1, wherein administration is chronic administration.

3. The method of claim 1, wherein said COX-1 selective inhibitor is provided at a dosage sufficient to inhibit platelet stimulated thromboxane production, but not sufficient to inhibit activated macrophage PGE2 production.

4. The method of claim 1, wherein said COX-1 selective inhibitor is SC560, SC58560, ketorolac, aspirin, valeryl salicylate, resveratrol, flurbiprofen, fenoprofen, sulindac, and piroxicam.

5. The method of claim 1, wherein inhibiting comprises blocking or reducing a diabetes-related increase in proteinuria and/or blocking or reducing a diabetes-related drop in glomerular filtration rate.

6. The method of claim 1, wherein treating comprises decreasing a diabetes-related proteinuria and/or increasing a diabetes-related depressed glomerular filtration rate.

7. The method of claim 1, wherein the administration is oral.

8. The method of claim 1, further comprising administering an angiotensin converting enzyme inhibitor.

9. The method of claim 1, wherein said subject suffers from type I diabetes.

10. The method of claim 1, wherein said subject suffers from type II diabetes.

11. An improved method of type I diabetes therapy comprising chronic administration to a diabetic subject of a composition comprising insulin and a cyclooxygenase (COX-1) selective inhibitor.

12. The method of claim 11, wherein the administration is intramuscular, intravenous, subcutaneous, intranasal, inhalational or transdermal.

13. The method of claim 11, wherein said COX-1 selective inhibitor is provided at a dose providing at least 2-fold greater activity against COX-1 and compared to COX-2.

14. The method of claim 11, wherein said COX-1 selective inhibitor is SC560, SC58560, ketorolac, aspirin, valeryl salicylate, resveratrol, flurbiprofen, fenoprofen, sulindac, and piroxicam.

15. A pharmaceutical formulation comprising insulin and a COX-1 selective inhibitor.

16. A pharmaceutical formulation comprising an ACE inhibitor and a COX-1 selective inhibitor.
